(19) United States
(12) Patent Application Publication
(10) Pub. No.: US 2014/0308364 A1
(43) Pub. Date: Oct. 16, 2014

(54) GELATINOUS HYDROXYAPATITE-NANOCOMPOSITES

(71) Applicant: Ching-Chang Ko, Chapel Hill, NC (US)

(72) Inventor: Ching-Chang Ko, Chapel Hill, NC (US)

(73) Assignee: THE UNIVERSITY OF NORTH CAROLINA AT CHAPEL HILL, Chapel Hill, NC (US)

(21) Appl. No.: 14/357,451

(22) PCT Filed: Nov. 15, 2012
(86) PCT No.: PCT/US2012/065216

§ 371 Date: May 9, 2014

Related U.S. Application Data
(60) Provisional application No. 61/560,777, filed on Nov. 16, 2011.

(51) Int. Cl. A61L 27/12; A61L 27/54

(52) U.S. Cl. CPC A61L 27/12; A61L 27/54
USPC 424/602

(57) ABSTRACT

A novel nanocomposite including hydroxyapatite-gelatin bioceramic material and dopamine, wherein the dopamine undergoes an oxidative self-polymerization reaction to form the novel nanocomposite. The nanocomposite displays superior mechanical strength, elasticity, biocompatibility and forming capabilities and is targeted for bone repairs and template-assisted tissue engineering applications. In addition, improved aminosilica-based hydroxyapatite-gelatin bioceramic bioceramics are disclosed.

### CROSS REFERENCE TO RELATED APPLICATIONS

This application claims priority to U.S. Provisional Patent Application No. 61/560,777 filed Nov. 16, 2011 in the name of Ching-Cheng KO entitled “Gelatinous Hydroxyapatite-Nanocomposites,” which is hereby incorporated by reference herein in its entirety.

### GOVERNMENT RIGHTS IN THE INVENTION

The United States Government has rights to this invention pursuant to National Institute of Health grant number DE018695.

### FIELD

This invention relates generally to the use of sol-gel based hydroxyapatite-gelatin bioceramic (GEMOSOL) and aminosilica-based hydroxyapatite-gelatin bioceramic (GEMOSIL) nanoparticles in the formation of new tissue engineering carriers for bone regeneration. More particularly, the present invention relates to polydopamine bio-inspired hydroxyapatite-gelatin nanocomposites (PDHG) and improved aminosilica-based hydroxyapatite-gelatin bioceramic bio ceramics.

### DESCRIPTION OF THE RELATED ART

Critical size defects in bone can be difficult to manage and may require multiple-phase surgery to achieve adequate reparation and function. Grafts are currently the most popular procedures in the United States for the repair of skeletal defects. Because a functional biomaterial that mimicks the natural bone's mechanical strength and composition is not currently available, only autografts (using the patient's own bone) and xenografts (using tissues from other species) are currently in clinical use. Disadvantageously, autogenous bone lacks formability, and this type graft is frequently associated with complications such as donor site availability and morbidity, infection, and malformation. A major problem with xenografts is immune rejection. Other grafting materials such as alloplastic grafts have been proposed and developed, however, they cannot provide enough mechanical strength to sustain loads. Ceramics have been proposed, however, hydroxyapatite ceramics are largely non-resorbable while amorphous calcium phosphate is too weak to restore defects.

A new approach to skeletal defects, which has potential to produce a paradigm shift in treatment of tissue defects and deficiencies, is tissue engineering (TE), which uses synthetic biomaterials to carry stem cells and growth factors into defect areas in an attempt to regenerate a permanent replacement. The synthetic biomaterial forms scaffolds serving as a template to guide the regeneration, but should eventually be replaced by the patient's own newly-formed bone or other tissue. Although many improvements have been made in biodegradable polymeric materials for use with skeletal defects and in calcium phosphate composites, there have been few efforts to advance toward bio-inspired hydroxyapatite-collagenous composites.

The present inventors previously created unique hydroxyapatite-gelatin nanocomposite particles (HAP-GEL) by co-precipitation processing as well as aminosilica-based hydroxyapatite-gelatin bioceramic nanoparticles (GEMOSIL). These rigid, biomimetic scaffolds of hydroxyapatite-gelatin (HAP-GEL) nanocomposites combine the advantages of strength and resorption from ceramics and polymers, respectively. The preliminary data via rat femur also suggest that the hydroxyapatite in HAP-GEL is resorbable, similar to natural bone. Although not wishing to be bound by theory, it is thought that each gelatin particle immobilizes a cluster of nano-crystal HAPs that were precipitated in situ onto gelatin with —COO⁻/Ca⁺² binding. One possible mechanism that may explain the resorbable property of HAP-GEL may be the cell binding of gelatin. In addition, the wetting ability of HAP-GEL particles significantly differs from conventional sintered HAP. The HAP-GEL instantaneously absorbs large amount of water while HAP does not, which renders HAP-GEL advantageous for processing and results in greater mechanical strengths when it interacts with various solvents (i.e., water and methanol).

HAP-GEL derived biomaterials may ultimately serve as a new TE carrier for bone regeneration in critical size defects in both craniofacial and other skeletal areas. Towards that end, the development of a polydopamine bio-inspired hydroxyapatite-gelatin nanocomposite (PDHG) is disclosed herein. The PDHG nanocomposites can be varied to provide different compressive strengths and can stimulate bone formation even without adding cells and growth factors. Advantageously, the PDHG formula can be optimized to alter the material's physical properties and formability.

### SUMMARY

The present invention relates generally to novel composite bioceramics. More specifically, the present invention relates to hydroxyapatite-gelatin formable bioceramics and methods of making and using same.

In one aspect, a method of making a GEMUSSEL nanocomposite is described, said method comprising combining hydroxyapatite-gelatin (HAP-GEL) bioceramic, at least one dopamine species, at least one oxidizing agent, and at least one calcium salt to form a GEMUSSEL material, wherein the GEMUSSEL material hardens to form the GEMUSSEL nano composite.

In another aspect, a method of making a GEMUSSEL nanocomposite is described, said method comprising combining hydroxyapatite-gelatin (HAP-GEL) bio ceramic, at least one dopamine species, at least one oxidizing agent, at least one silane reactant, and at least one calcium salt to form a GEMUSSEL material, wherein the GEMUSSEL material hardens to form the GEMUSSEL nano composite.

In still another aspect, a polydopamine bio-inspired hydroxyapatite-gelatin nanocomposites (PDHG) nanocomposite comprising dopamine and hydroxyapatite-gelatin nanocrystals is described.

In yet another aspect, a method of making an aminosilica-based hydroxyapatite-gelatin bioceramic is described, said method comprising combining hydroxyapatite-gelatin (HAP-GEL) bioceramic, at least one silane reactant, at least one calcium salt, and at least one accelerator material to form the GEMOSIL2 material, wherein the GEMOSIL2 material hardens to form the GEMOSIL2 bioceramic.

Other aspects, features and embodiments will be more fully apparent from the ensuing disclosure and appended claims.

### BRIEF DESCRIPTION OF THE DRAWINGS

FIG. 1 is a schematic of an embodiment of the method of making the PDHG nanocomposite (GEMUSSEL).

FIG. 2 is a schematic of a second embodiment of the method of making the PDHG nano composite (GEMUSSEL).

FIG. 3 is a schematic of a method of making the GEMOSIL2 bioceramic.

FIG. 4 illustrates the injectability of the PDHG paste.

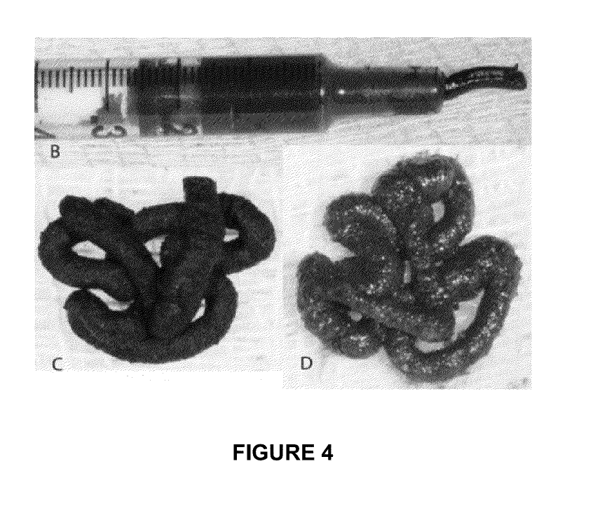

### DETAILED DESCRIPTION, AND PREFERRED EMBODIMENTS THEREOF

The present invention relates generally to polydopamine bio-inspired hydroxyapatite-gelatin nanocomposites (PDHG), and more particularly PDHG nanocomposites that are made using sol-gel based hydroxyapatite-gelatin bioceramic (GEMOSOL) and/or aminosilica-based hydroxyapatite-gelatin bioceramic (GEMOSIL) nanocrystals. In addition, the present invention relates to an improved GEMOSIL bioceramic, referred to as GEMOSIL2.

The PDHG nanocomposites, also referred to GEMUSSEL, described herein rely on two bio-inspired principles: 1) self-organization of HAP nanocrystals along the gelatin fibrils by chemical interaction between calcium ions of hydroxyapatite (HAP) and the carboxyl groups of the gelatin molecules, and 2) pH-induced calcium-ligand cross-links inspired by dopamine, whereby the catechol groups in dopamine form hydrogen bonds, metal-ligand complexes and quinhydrone charge-transfer complexes which provide adhesion to the HAP-GEL. By using dopamine self-polymerization and catechol-calcium chelates, the GEMUSSEL undergo bulk solidification. The GEMUSSEL is effective in promoting healing bone growth and formation even without cells and stimulation factors. As a consequence, an efficacious bone regeneration based on PDHG results.

The present inventors previously described sol-gel based hydroxyapatite-gelatin bioceramic (GEMOSOL) and aminosilica-based hydroxyapatite-gelatin bioceramic (GEMOSIL) nanoparticles in U.S. patent application Ser. No. 12/685,743 having a filing date of Jan. 12, 2010 in the name of Luo et al., which is hereby incorporated herein in its entirety. In addition, the present inventors described a biomimetic nanocomposite including hydroxyapatite nanocrystals, gelatin, and polymer, wherein the biomimetic nanocomposite is crosslinked in U.S. patent application Ser. No. 11/305,663 having a filing date of Dec. 16, 2005, which is also incorporated herein in its entirety. In summary, the GEMOSOL nanoparticles comprised hydroxyapatite nanocrystals, gelatin and sol-gel-containing materials. The GEMOSIL nanoparticles comprised hydroxyapatite nanocrystals, gelatin and aminosilica-containing material. Advantageously, sol-gel based biomaterials can be synthesized from solutions at room temperature, which makes these biomaterials suitable for the incorporation of biomolecules and living cells for biomedical applications. The sol-gel process is a wet-chemical technique whereby a chemical solution undergoes hydrolysis and polycondensation reactions to produce colloidal particles (i.e., a including particles ranging in size from from 1 nm to 1 μm) (the “sol”) such as metal oxides. The sol will form an inorganic network containing a liquid phase (the “gel”). The “sol-gel” materials, as defined herein, include SiO₂, TiO₂, ZrO₂, and combinations thereof.

As defined herein, “substantially dispersed” and “substantially uniformly dispersed” corresponds to less than 10% variation in the chemical makeup throughout the composite, regardless of whether sampled interiorly or exteriorly, preferably less than 5% variation, and most preferably less than 2% variation.

As defined herein, “silica” corresponds to SiO₂.

For the purposes of this disclosure, HAP-GEL is used to generally describe the GEMOSOL or GEMOSIL particles, as well as any other hydroxyapatite-gelatin nanocomposite particles known in the art.

### THE PRIOR ART

Methods for producing the HAP-GEL nanoparticles were previously described in U.S. patent application Ser. No. 12/685,743. Suitable gelatins include both high bloom and low bloom gelatin. Preferably, gelatins having a bloom value between about 100 and about 300 will be used. “Bloom value” is a measurement of the strength of a gel formed by a 6 and ⅔% solution of the gelatin, that has been kept in a constant temperature bath at 10 degrees centigrade for 18 hours. The properties of the final HAP-GEL nanoparticle depend in part on the characteristics of the gelatin used. Variously, gelatin may be obtained that is produced from different animals, including cows and pigs. Gelatin may be extracted from various collagen-containing body parts, including bone and skin. The gelatin may be selected according to the desired application, as different gelatins, depending on the source and the extent of denaturation, may provide a better choice for the composite, depending upon the desired mechanical properties or biological activity level. Generally, it has been found that bovine gelatin provides better composites for many applications. An example of a suitable gelatin is standard unflavored gelatin (available from Natural Foods Inc., Canada). The gelatin may be dissolved into solution before use, preferably to form an aqueous solution. The gelatin may be used without purification or other prepatory steps.

The gelatin may be modified prior to use in a reaction mixture. Preferably, the gelatin will be at least partially phosphorylated before use as a reactant. For example, the gelatin may be phosphorylated by the addition of phosphoric acid, ammonium phosphate ((NH₄)₃PO₄), diammonium hydrogen phosphate ((NH₄)₂HPO₄), ammonium dihydrogen phosphate (NH₄H₂PO₄), monoammonium phosphate (NH₄.H₂PO₄), or combinations thereof (available from chemical supply firms such as Fisher Scientific and Sigma Chemical) to a gelatin solution, or the gelatin may be added to a phosphoric acid solution. It is believed that phosphorylation leads to and enables better dispersion and growth of the hydroxyapatite nanocrystals. In solutions with phosphorylated gelatin, there will typically be excess phosphoric acid available for later crystal formation and/or growth.

The hydroxyapatite nanocrystals are formed through a reaction between phosphoric acid and/or phosphorylated locations on the gelatin fibers and calcium hydroxide. The phosphorylated locations are frequently the starting locations for hydroxyapatite crystal growth, however, hydroxyapatite crystal growth may also occur in solution between the phosphoric acid and calcium hydroxide components. These crystals may grow and embed themselves into the gelatin matrix structure by binding themselves to groups, such as carboxyl and amide groups, on the gelatin molecules. Once begun, the crystals grow by incorporating more calcium hydroxide and phosphoric acid components into the crystal. The product of this reaction includes a co-precipitated hydroxyapatite-gelatin colloidal material.

Calcium hydroxide is available from chemical supply firms such as Fisher Scientific and Sigma Chemical. However, calcium hydroxide may also be produced in a process including calcining calcium carbonate, which removes carbon dioxide to form calcium oxide. After calcining, the calcium oxide is hydrated to form calcium hydroxide. Following hydration, the calcium hydroxide may be weighed as a quality check. Due to the reactive nature of calcium hydroxide, and the tendency of calcium hydroxide to degrade quickly, special care should be taken with calcium hydroxide to ensure a high quality level of the calcium hydroxide. Because of this concern with the quality of the calcium hydroxide, producing calcium hydroxide just prior to use is preferred.

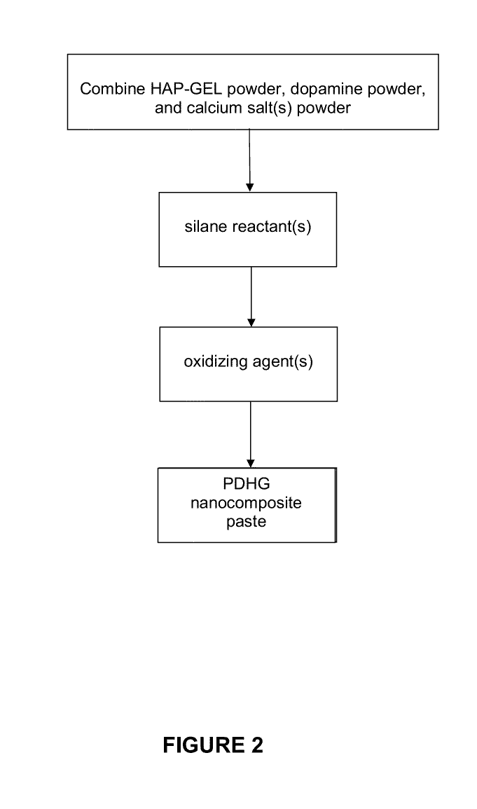

The hydroxyapatite-gelatin colloid may be incorporated into a sol-gel or silica matrix with or without removable active fillers and/or other additives to produce the formable bioceramic described herein, as shown schematically in FIG. 2. Although not wishing to be bound by theory, it is thought that the hydroxyapatite-gelatin colloid at least partially dissolves in the sol-gel or silica matrix, which creates a strong bond. Silane reactants contemplated for the sol-gel or silica matrix include, but are not limited to, tetramethylorthosilicate (TMOS), tetraethylorthosilicate (TEOS), 3-aminopropyltrimethoxysilane, bis[3-(trimethoxysilyepropyl]-ethylenediamine, bis[3-(triethoxysilyepropyl]-ethylenediamine, methyltrimethoxysilane (MTMS), polydimethylsilane (PDMS), propyltrimethoxysilane (PTMS), methyltriethoxysilane (MTES), ethyltriethoxysilane, dimethyldiethoxysilane, diethyldiethoxysilane, diethyldimethoxysilane, bis(3-trimethoxysilylpropyl)-N-methylamine, 3-(2-Aminoethylamino)propyltriethoxysilane, N-propyltriethoxysilane, 3-(2-Aminoethylamino)propyltrimethoxysilane, methylcyclohexyldimethoxysilane, dimethyldimethoxysilane, dicyclopentyldimethoxysilane, 3-[2(vinylbenzylamino)ethylamino]propyltrimethoxysilane, 3-aminopropyltriethoxysilane, 3-(aminopropyl)dimethylethoxysilane, 3-(aminopropyl)methyldiethoxysilane, 3-(aminopropyl)methyldimethoxysilane, 3-(aminopropyl)dimethylmethoxysilane, N-butyl-3-aminopropyltriethoxysilane, N-butyl-3-aminopropyltrimethoxysilane, N-(β-amimoethyl)-γ-amino-propyltriethoxysilane, 4-amino-butyldimethyl ethoxysilane, N-(2-Aminoethyl)-3-aminopropylmethyldimethoxysilane, N-(2-Aminoethyl)-3-aminopropylmethyldiethoxysilane, 3-aminopropylmethyldiethoxysilane, or combinations thereof. Preferably, the silane reactant includes at least one amino-containing silane reactant, more preferably bis[3-(trimethoxysilyepropyl]-ethylenediamine (enTMOS). Titanium reactants contemplated for the sol-gel matrix include, but are not limited to, titanium isopropoxide. Zirconium reactants contemplated for the sol-gel matrix include, but are not limited to, zirconium ethoxide, zirconium propoxide, and zirconium oxide.

Inactive filler material includes, but is not limited to, poly(lactic-co-glycolic acid), poly(lactic acid), poly(glycolic acid), polyacrylic acid, poly(ethylene oxide), poly(methyl methacrylate), calcium phosphate, potassium chloride, calcium carbide, calcium chloride, sodium chloride, polystyrene, and combinations thereof. Some inactive fillers can be solidified with the GEMOSIL nanocomposite to serve as structural templates including, but not limited to, poly(N-isopropylacrylamide) and calcium chloride. Poly(N-isopropylacrylamide) may be removed from the bioceramic following formation of same by lowering the incubation temperature.

In general, a reactor is setup with temperature control and stirring. A mixture of calcium hydroxide, phosphoric acid, and gelatin is mixed together using a high degree of agitation. These components should be as pure as possible to minimize any contaminants which might weaken the resulting bioceramic. Purchased or produced, the components will preferably be placed into solution prior to use. More preferably, the components will be in an aqueous solution. The various components may be added all at once, or may be added gradually. If added gradually the components in solution may be added using pumps, such as peristaltic pumps (such as Masterflex, available from Cole-Parmer).

The gelatin may be added separately, or alternatively, may be pre-mixed together with one of the other components prior to addition. Preferably, the gelatin will be pre-mixed with the phosphoric acid in order to at least partially phosphorylate the gelatin. In order to assist in dissolving the mixture, the temperature may be controlled between about 35° C. and 40° C., and the mixture stirred during the addition and dissolving. A wide range of gelatin concentrations may be used. Preferably, the concentration will be greater than about 0.001 mmol, greater than about 0.01 mmol, or greater than about 0.025 mmol Preferably, the concentration will be 100 mmol or less, 10 mmol or less, or 1 mmol or less. In order to enable sufficient phosphorylation of the gelatin, this mixing should continue for some time. Suitably, the mixing will continue for at least about 2 hours to about 24 hours.

After preparation, the calcium, phosphoric acid, and gelatin components (or calcium, phosphorylated gelatin, and optionally additional phosphoric acid) are added together, using agitation and while controlling the pH and temperature. Suitably, the pH will be controlled to be greater than about 7.0 but less than 9.0. The temperature of the mixture may be controlled to be greater than about 30° C. and less than about 48° C. As the components streams are added, co-precipitation begins to occur. This co-precipitation results in the formation of hydroxyapatite nanocrystals in and/or on the gelatin. Preferably, the conditions and component concentrations are maintained such that the continued high-speed agitation and controlled conditions result in the continued formation of hydroxyapatite nanocrystals, rather than the growth of macro-crystals. Under high agitation, this mixture forms a colloidal slurry.

Properly controlled, the co-precipitation results in a uniform dispersion of hydroxyapatite nanocrystals. Preferably, the ratio of the number of moles of calcium to the number of moles of phosphate present (as free phosphate and/or phosphorylated gelatin) will be from about 1.5 to about 2.0, more preferably present in a ratio from about 1.6 to about 1.75, and most preferably from about 1.65 to about 1.70. The nanocrystals formed may be needle-shaped, plate-shaped, or may have other crystal shapes. Preferably, hydroxyapatite crystals formed will be needle-shaped.

After addition of all of the components into the co-precipitation reaction, agitation is stopped. Following solidification, water may be removed from the hydroxyapatite-gelatin biomaterial. For example, the slurry can be centrifuged to remove excess water, preferably at temperatures below about 10° C. for time in a range from about 10 min to about 60 min. The hydroxyapatite-gelatin material may be dried using methods known to those skilled in the art. For example, the hydroxyapatite-gelatin biomaterial can be freeze dried, lyophilized or both. Once dry the HAP-GEL can be ground into powder, preferably in a range from about 100 μm to about 300 μm in size. When the hydroxyapatite-gelatin bioceramic is sol-gel based it is referred to as a GEMOSOL bioceramic and when the hydroxyapatite-gelatin bioceramic is aminosilica-based it is referred to as a GEMOSIL bioceramic.

### The PDHG Nanocomposite (GEMUSSEL)

In a first aspect, a method of making polydopamine bio-inspired hydroxyapatite-gelatin nanocomposites (PDHG) is described, said method comprising the combination of HAP-GEL nanoparticles with dopamine and other additives. Although not wishing to be bound by theory, the combination of the HAP-GEL nanoparticles with dopamine and other additives is thought to result in the self-polymerization of dopamine to form the polydopamine bio-inspired hydroxyapatite-gelatin nanocomposites (PDHG), otherwise referred to as GEMUSSEL. Advantageously, dopamine (4-(2-aminoethyl)benzene-1,2-diol) is biosynthesized in the body and hence, the GEMUSSEL will have better resorption and osteoconductivity relative to other TE products proposed to date.

In one embodiment, the method of making GEMUSSEL comprises combining HAP-GEL, at least one dopamine species, at least one oxidizing agent, and at least one calcium salt to form the GEMUSSEL material, wherein the GEMUSSEL material hardens to form the GEMUSSEL. By adjusting the amount of water in the system, the setting time can be varied and the GEMUSSEL nanocomposite injectable. In one embodiment, the method of making GEMUSSEL comprises combining HAP-GEL, at least one dopamine species, ammonium persulfate, and at least one calcium salt to form the GEMUSSEL material, wherein the GEMUSSEL material hardens to form the GEMUSSEL nanocomposite. In another embodiment, the method of making GEMUSSEL comprises combining HAP-GEL, at least one dopamine species, at least one oxidizing agent, and Ca(OH)₂ to form the GEMUSSEL material, wherein the GEMUSSEL material hardens to form the GEMUSSEL nanocomposite. In yet another embodiment, the method of making GEMUSSEL comprises combining HAP-GEL, at least one dopamine species, at least one oxidizing agent, and at least two calcium salts to form the GEMUSSEL material, wherein the GEMUSSEL material hardens to form the GEMUSSEL nanocomposite. In still another embodiment, the method of making GEMUSSEL comprises combining HAP-GEL, at least one dopamine species, at least one oxidizing agent, CaO and Ca(OH)₂ to form the GEMUSSEL material, wherein the GEMUSSEL material hardens to form the GEMUSSEL nanocomposite. In another embodiment, the method of making GEMUSSEL comprises combining HAP-GEL, at least one dopamine species, ammonium persulfate, CaO and Ca(OH)₂ to form the GEMUSSEL material, wherein the GEMUSSEL material hardens to form the GEMUSSEL nanocomposite. The compressive strength of the GEMUSSEL is preferably in a range from about 10 MPa to about 170 MPa.

Alternatively, the method of making GEMUSSEL comprises combining HAP-GEL, at least one dopamine species, at least one oxidizing agent, at least one silane reactant, and at least one calcium salt to form the GEMUSSEL material, wherein the GEMUSSEL material hardens to form the GEMUSSEL. In one embodiment, the method of making GEMUSSEL comprises combining HAP-GEL, at least one dopamine species, ammonium persulfate, at least one silane reactant, and at least one calcium salt to form the GEMUSSEL material, wherein the GEMUSSEL material hardens to form the GEMUSSEL nanocomposite. In another embodiment, the method of making GEMUSSEL comprises combining HAP-GEL, at least one dopamine species, at least one oxidizing agent, at least one silane reactant, and Ca(OH)₂ to form the GEMUSSEL material, wherein the GEMUSSEL material hardens to form the GEMUSSEL nanocomposite. In yet another embodiment, the method of making GEMUSSEL comprises combining HAP-GEL, at least one dopamine species, at least one oxidizing agent, bis[3-(trimethoxysilyl)propyl]-ethylenediamine (enTMOS), and at least one calcium salt to form the GEMUSSEL material, wherein the GEMUSSEL material hardens to form the GEMUSSEL nanocomposite. By adjusting the amount of water in the system, the setting time can be varied and the GEMUSSEL nanocomposite injectable. For example, a component may have water in it (e.g., an oxidizing agent). Alternatively, or in addition to, some additional water can be added. Most preferably, the method of making GEMUSSEL is carried out without any added water, i.e., the water is naturally part of at least one component. The compressive strength of the GEMUSSEL is preferably in a range from about 10 MPa to about 170 MPa.

The PDHG nanocomposites (GEMUSSEL) can be prepared by sequentially combining HAP-GEL powders, at least one calcium salt powder, dopamine powder, and at least one oxidizing agent. It should be appreciated that the order of addition can be altered, e.g., the HAP-GEL powders, the at least one calcium salt powder, the dopamine, and at least one oxidizing agent can be added in any order, as readily understood by the person skilled in the art. Alternatively, the PDHG nanocomposites (GEMUSSEL) can be prepared by sequentially combining HAP-GEL powders, at least one calcium salt powder, dopamine powder, at least one silane reactant, and at least one oxidizing agent. It should be appreciated that the order of addition can be altered, e.g., the HAP-GEL powders, the at least one calcium salt powder, the dopamine powder, at least one silane reactant, and at least one oxidizing agent can be added in any order, as readily understood by the person skilled in the art. It should be appreciated that HAP-GEL powders can be the prepared using the methods described hereinabove (previously described in U.S. patent application Ser. No. 12/685,743) or by any other HAP-GEL powder known in the art, including the GEMOSIL2 described herein. Following the combination of the components, depending on the ratio of components, the GEMUSSEL material will be a viscous paste that will harden in about 1 minute to about 30 minutes, more preferably about 5 minutes to about 15 minutes. The hardening time can also controlled by using a cold stage and mold (−70° C. to 50° C.). Moreover, the paste is injectable and is easily delivered using a syringe.

Calcium salts contemplated herein include, but are not limited to, calcium oxide, calcium hydroxide, calcium carbonate, calcium nitrate, calcium phosphate, calcium fluoride, calcium chloride, calcium iodide, calcium oxalate, calcium citrate, calcium pyrophosphate, and any combination thereof. Preferably, the at least one calcium salt comprises calcium oxide, calcium hydroxide, or a combination of calcium oxide and calcium hydroxide.

The oxidizing agents are preferably water soluble and include, but are not limited to, hydrogen peroxide (H₂O₂), ferric nitrate (Fe(NO₃)₃), potassium iodate (KIO₃), nitric acid (HNO₃), ammonium chlorite (NH₄ClO₂), ammonium chlorate (NH₄ClO₃), ammonium iodate (NH₄IO₃), ammonium perborate (NH₄BO₃), ammonium perchlorate (NH₄ClO₄), ammonium periodate (NH₄IO₃), ammonium persulfate ((NH₄)₂S₂O₈), tetramethylammonium chlorite ((N(CH₃)₄)ClO₂), tetramethylammonium chlorate ((N(CH₃)₄)ClO₃), tetramethylammonium iodate ((N(CH₃)₄)IO₃), tetramethylammonium perborate ((N(CH₃)₄)BO₃), tetramethylammonium perchlorate ((N(CH₃)₄)CIO₄), tetramethylammonium periodate ((N(CH₃)₄)IO₄), tetramethylammonium persulfate ((N(CH₃)₄)S₂O₈), urea hydrogen peroxide ((CO(NH₂)₂)H₂O₂), and combinations thereof. Preferably, the at least one oxidizing agent comprises ammonium persulfate. Although not wishing to be bound by theory, it is thought that the oxidizing agent speeds up the dopamine self-polymerization reaction.

Dopamine is well known in the art and has the chemical formula 4-(2-aminoethyl)benzene-1,2-diol. It is known that dopamine undergoes oxidative self-polymerization onto surfaces, which was originally inspired by the adhesive properties displayed by mussels (Lee et al., Science, 318, 426-430 (2007)). In addition to dopamine, functionalized dopamine monomers can be used including carboxylic functionalized dopamine, hydroxy functionalized dopamine, and thiol functionalized dopamine. Dopamine, or a derivative thereof, causes the HAP-GEL nanocrystals throughout the matrix to be glued together by polydopamine. In addition, the polydopamine appears to form nano-networks within the siloxane matrix, resulting in a silica structure with greater degradability than compounds without polydopamine. Adding dopamine, which forms polymeric chains, doubles tensile (biaxial flexure) strength as well as increases degradation and decreases the rate of calcium leaching.

Silane reactants contemplated for the sol-gel or silica matrix include, but are not limited to, tetramethylorthosilicate (TMOS), tetraethylorthosilicate (TEOS), 3-aminopropyltrimethoxysilane, bis[3-(trimethoxysilyl)propyl]-ethylenediamine, bis[3-(triethoxysilyl)propyl]-ethylenediamine, methyltrimethoxysilane (MTMS), polydimethylsilane (PDMS), propyltrimethoxysilane (PTMS), methyltriethoxysilane (MTES), ethyltriethoxysilane, dimethyldiethoxysilane, diethyldiethoxysilane, diethyldimethoxysilane, bis(3-trimethoxysilylpropyl)-N-methylamine, 3-(2-Aminoethylamino)propyltriethoxysilane, N-propyltriethoxysilane, 3-(2-Aminoethylamino)propyltrimethoxysilane, methylcyclohexyldimethoxysilane, dimethyldimethoxysilane, dicyclopentyldimethoxysilane, 3-[2(vinylbenzylamino)ethylamino]propyltrimethoxysilane, 3-aminopropyltriethoxysilane, 3-(aminopropyl)dimethylethoxysilane, 3-(aminopropyl)methyldiethoxysilane, 3-(aminopropyl)methyldimethoxysilane, 3-(aminopropyl)dimethylmethoxysilane, N-butyl-3-aminopropyltriethoxysilane, N-butyl-3-aminopropyltrimethoxysilane, N-(β-amimoethyl)-γ-amino-propyltriethoxysilane, 4-amino-butyldimethyl ethoxysilane, N-(2-Aminoethyl)-3-aminopropylmethyldimethoxysilane, N-(2-Aminoethyl)-3-aminopropylmethyldiethoxysilane, 3-aminopropylmethyldiethoxysilane, or combinations thereof. Preferably, the silane reactant includes at least one amino-containing silane reactant, more preferably bis[3-(trimethoxysilyepropyl]-ethylenediamine (enTMOS).

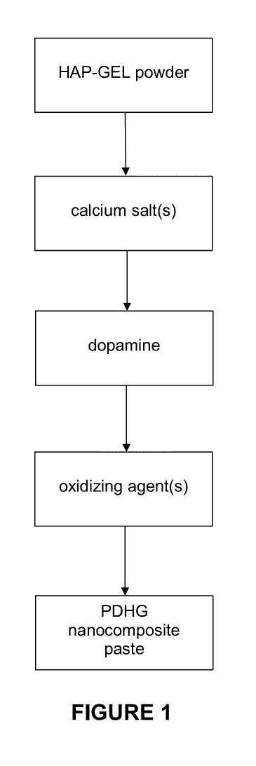

In one embodiment, a mixture of Ca(OH)₂ and CaO, having a weight ratio of Ca(OH)₂/CaO of about 1:10 to about 10:1, preferably about 1:2 to about 2:1, is combined with HAP-GEL powder to form a calcium salt(s)/HAP-GEL powder mixture. The weight ratio of calcium salt(s)/HAP-GEL powder is about 1:10 to about 10:1, preferably about 1:2 to about 3:1. The powders are mixed to preferentially form a substantially homogeneous mixture. In a separate container, dopamine is dissolved an acid/alcohol solvent. Preferably the acid is a mineral acid such as hydrochloric acid, nitric acid, or phosphoric acid, preferably HCl, and the concentration of mineral acid is about 0.1 to about 2 N, preferably about 0.5 to about 1.5 N, in alcohol. The alcohol can be a monohydric alcohol such as branched or straight-chained C₁-C₃ alkanols (i.e., methanol, ethanol, propanol) or diols (i.e., ethylene glycol, propylene glycol). For example, dopamine can be dissolved in an 1 N HCl in methanol solution. The calcium salt/HAP-GEL powder mixture is mixed with the dopamine mixture to form a third mixture. The weight ratio of calcium salt/HAP-GEL powder mixture relative to dopamine mixture is about 1:10 to about 10:1, preferably about 1:2 to about 6:1. Thereafter, the at least one oxidizing agent, preferably ammonium persulfate in water, is added to the third mixture to form the GEMUSSEL nanocomposite paste, wherein the ratio of the third mixture/oxidizing agent is about 1:10 to about 100:1, preferably about 1:1 to about 30:1. The GEMUSSEL nanocomposite paste can be pressed into a mold and will harden to form the GEMUSSEL nanocomposite. This embodiment is illustrated in FIG. 1. The temperature of the method is in a range from about 15° C. to about 50° C., preferably about 20° C. to about 37° C., most preferably room temperature. The nanocomposite can be dehydrated in air at room temperature.

In another embodiment, the method of making GEMUSSEL comprises combining HAP-GEL powder, dopamine powder, and at least one calcium salt powder, followed by the addition of the at least one silane reactant, followed by the addition of the at least one oxidizing agent, as depicted in FIG. 2. For example, HAP-GEL powder, dopamine powder and Ca(OH)₂ powder can be combined and mixed. Thereafter a silane reactant such as an amino-containing silane, e.g., enTMOS, can be added to the powder mixture. Lastly, at least one oxidizing agent in water, such as a persulfate, e.g., ammonium persulfate, is added to the third mixture to form the GEMUSSEL nanocomposite paste. The GEMUSSEL nanocomposite paste can be pressed into a mold and will harden to form the GEMUSSEL nanocomposite. This embodiment is illustrated in FIG. 1. The temperature of the method is in a range from about −70° C. to about 50° C., preferably about −20° C. to about 37° C., most preferably room temperature. The nanocomposite can be dehydrated in air at room temperature.

Interestingly, varying the amount of water in the method affects the viscosity of the GEMUSSEL nanocomposite paste. Water can be “added” during the method of making the GEMUSSEL nanocomposite by dissolving the dopamine therein as well as the oxidizing agent. By varying the amount of water, the GEMUSSEL paste can be formulated to be thicken and eventually be injectable.

In another embodiment, instead of dopamine, a dopamine-graft polymer is used to increase the GEMUSSEL nanocomposite's toughness. For example, it is contemplated that dopamine could be grafted on a polymer comprising poly-L-Lactide (PLLA), poly-trimethylene carbonate (PTMC) and/or polycarbonate (PC), such as P(LLA-co-PC) co-polymer.

Advantages associated with the novel GEMUSSEL nanocomposites described herein include, but are not limited to, compatibility with carbon-based lifeforms, good mechanical strength, excellent compressive strength, superb formability for scaffolding and upregulated cell differentiation.

Optionally, other components or additives may be added to the GEMUSSEL nanocomposite. These additives may be added for various reasons. For example, additives may be added to increase biocompatibility, to decrease the possibility of rejection, to decrease the risk of infection, to increase the rate of natural bone growth in the GEMUSSEL nanocomposite, or to increase the rate of natural cell growth near the implant. Additives may also be added to change or enhance some of the properties of the GEMUSSEL nanocomposite. For example, the GEMUSSEL nanocomposite may include growth factors, cells, other materials and elements, curing or hardening components, and other possible additives. Examples of suitable growth factors include, but are not limited to, bone morphogenic protein (BMP), transforming growth factor (TGF-β), vascular endothelial growth factor (VEGF), matrix gla protein (MGP), bone siloprotein (BSP), osteopontin (OPN), osteocacin (OCN), insulin-like growth factor (IGF-I), Biglycan, Receptor activator of nuclear factor kappa B ligand (RANKL), dexmethasone, nitrogen oxide, TGF-β1, and procollagen type I (Pro COL-α1). Suitable cells include, but are not limited to, osteoblasts, osteoclasts, osteocytes, mesenchymal stem cells (MSC), multipotent stem cells, embryonic stem cells (ESC), and induced pluripotent stem cells (IPS). Other materials or elements that can be added include titanium-containing materials such as TiO₂.

The GEMUSSEL nanocomposite may be used for a wide range of alloplastic uses, for a variety of purposes, and in a variety of applications. Alloplastic refers to synthetic biomaterials, in contrast to natural biomaterials which may be from the same individual (autogenic), from the same species (allogenic), or from a different species (xenogenic). The properties of the GEMUSSEL nanocomposite may be modified to better meet the requirements of the use, purpose, or application for which it is intended. The properties depend in part on the gelatin used, the stoichiometry of the HAP-GEL, the amount and type of silane reactant(s) used, the calcium salt ratio, the dopamine used for self-polymerization, the silane reactant, and the stoichiometry of the components of the GEMUSSEL nanocomposite. Thus, the resulting GEMUSSEL nanocomposite may have a wide range of mechanical properties.

These various properties lead to the ability of the GEMUSSEL nanocomposite to be used in a wide range of tissue engineering applications. For example, the GEMUSSEL nanocomposite can be made in scaffolds, which can deliver cells, growth factors, and other additives to a healing site. This can be used to regenerate bone, cartilage, and other tissues. Nano-scaled microstructures can be used to promote cell attachment, growth, and differentiation. Alternatively, the GEMUSSEL nanocomposite may be used to engineer alloplastic grafts. Thus, tissue engineering may be used to replace or augment many natural body tissues. Tissues may be regenerated using these types of structures, and additives may be used to compensate for deficiencies in the patient. Other structures that promote the rapid integration of the GEMUSSEL nanocomposite with the natural tissues may also be used effectively. For example, a structure of the GEMUSSEL nanocomposite may be implanted into a bone, which then acts to stimulate bone regeneration, especially in critical size defects in craniofacial and other skeletal areas. As another example, the GEMUSSEL nanocomposite may be implanted for cartilage replacement, which may stimulate cartilage regeneration. Another example is to use the GEMUSSEL nanocomposite for root canal fillers that will enhance tissue healing or regeneration. Still another example is to use the GEMUSSEL nanocomposite as an adhesive agent for dental applications.

The GEMUSSEL nanocomposite may be produced in different forms, depending upon the intended use and purpose. Suitable forms include solid, putty, paste, and liquid. If the GEMUSSEL nanocomposite is in solid form, it may be, for example, a shaped or unshaped solid, it may be a pre-formed solid, it may be a frame or a lattice, or another solid form. The GEMUSSEL nanocomposite may be formed into a porous scaffold. The solid form may be very stiff, stiff, slightly flexible, soft, rubbery, or other. The GEMUSSEL nanocomposite may be a putty. If in putty form, it may be anywhere from a dense or thin putty. The GEMUSSEL nanocomposite may be a paste. If a paste, it may be anywhere from a thick to a thin paste. If a liquid, it may be from very viscous to very thin.

Due to the fact that the GEMUSSEL nanocomposite can be formulated to thicken and hence be injectable, the GEMUSSEL nanocomposite lends itself to a wide range of uses. Uses of the GEMUSSEL nanocomposite include, but are not limited to: for bones, such as for bone graft material, bone cement, or bone replacement; for dental procedures, such as for dental implants, fillings, jaw strengthening or tooth replacement; for joint replacement; for cartilage replacement or reinforcement; for tendon or ligament replacement or repair; and a wide range of tissue engineering applications, including assisting in regenerating bodily tissues.

In a second aspect, a polydopamine bio-inspired hydroxyapatite-gelatin nanocomposites (PDHG) (also called GEMUSSEL) is disclosed, said nanocomposite being produced using the method of the first aspect. The GEMUSSEL nanocomposite comprises dopamine and HAP-GEL nanocrystals and prior to hardening, is an injectable material. The compressive strength of the GEMUSSEL nanocomposite is preferably in a range from about 10 MPa to about 170 MPa.

### Improved GEMOSIL

In a third aspect, a method of making a second generation of aminosilica-based hydroxyapatite-gelatin bioceramic (GEMOSIL2) is described, said method comprising combining powdered HAP-GEL, at least one silane reactant, at least one calcium salt, and at least one accelerator material to form the GEMOSIL2 material, wherein the GEMOSIL2 material hardens to form the GEMOSIL2 bioceramic. In one embodiment, the method of making the second generation aminosilica-based hydroxyapatite-gelatin bioceramic comprises combining powdered HAP-GEL, Ca(OH)₂, at least one silane reactant, and at least one accelerator material to form the GEMOSIL2 material, wherein the GEMOSIL2 material hardens to form the GEMOSIL2 bioceramic. In yet another embodiment, the method of making the second generation aminosilica-based hydroxyapatite-gelatin bioceramic comprises combining powdered HAP-GEL, at least two calcium salts, at least one silane reactant, and at least one accelerator material to form the GEMOSIL2 material, wherein the GEMOSIL2 material hardens to form the GEMOSIL2 bioceramic. In still another embodiment, the method of making the second generation aminosilica-based hydroxyapatite-gelatin bioceramic comprises combining powdered HAP-GEL, CaO, Ca(OH)₂, at least one silane reactant, and at least one accelerator material to form the GEMOSIL2 material, wherein the GEMOSIL2 material hardens to form the GEMOSIL2 bioceramic. In another embodiment, the method of making the second generation aminosilica-based hydroxyapatite-gelatin bioceramic comprises combining powdered HAP-GEL, CaO, Ca(OH)₂, at least one silane reactant, and at least one accelerator material to form the GEMOSIL2 material, wherein the GEMOSIL2 material hardens to form the GEMOSIL2 bioceramic. In still another embodiment, the method of making the second generation aminosilica-based hydroxyapatite-gelatin bioceramic comprises combining powdered HAP-GEL, CaO, Ca(OH)₂, at enTMOS, and at least one accelerator material to form the GEMOSIL2 material, wherein the GEMOSIL2 material hardens to form the GEMOSIL2 bioceramic. The compressive strength of the GEMOSIL2 bioceramic is preferably in a range from about 80 MPa to about 170 MPa. Preferably, the GEMOSIL2 material hardens to form the GEMOSIL2 bioceramic in about 1 min to about 30 min. Other materials or elements that can be added include titanium-containing materials such as TiO₂ to improve osteogenic property for bone regeneration.

The HAP-GEL used in the GEMOSIL2 bioceramic can include the sol-gel based hydroxyapatite-gelatin bioceramic (GEMOSOL) and/or aminosilica-based hydroxyapatite-gelatin bioceramic nanoparticles (GEMOSIL) described in U.S. patent application Ser. No. 12/685,743, or any other HAP-GEL bioceramic known in the art. The HAP-GEL is preferably dried and ground into a powder in a range from about 100 μm to about 300 μm prior to use in the method of making the GEMOSIL2 bioceramics. Preferably, the HAP-GEL used comprises GEMOSIL or GEMOSIL2 nanoparticles, preferably comprising the enTMOS silane compound.

Accelerator materials includes, but are not limited to, poly(lactic-co-glycolic acid), poly(lactic acid), poly(glycolic acid), polyacrylic acid, poly(ethylene oxide), calcium phosphate, potassium chloride, calcium carbide, calcium chloride, sodium chloride, polystyrene, and combinations thereof. Some accelerator materials can be solidified with the GEMOSIL2 bioceramic to serve as structural pore templates including, but not limited to, poly(N-isopropylacrylamide) and calcium chloride. Poly(N-isopropylacrylamide) may be removed from the bioceramic following formation of samples by lowering the incubation temperature. Preferably, the accelerator material comprises polyacrylic acid or calcium chloride.

Calcium salts contemplated herein include, but are not limited to, calcium oxide, calcium hydroxide, calcium carbonate, calcium nitrate, calcium phosphate, calcium fluoride, calcium chloride, calcium iodide, calcium oxalate, calcium citrate, calcium pyrophosphate, and any combination thereof. Preferably, the at least one calcium salt comprises calcium oxide, calcium hydroxide, or a combination of calcium oxide and calcium hydroxide.

Silane reactants contemplated include, but are not limited to, tetramethylorthosilicate (TMOS), tetraethylorthosilicate (TEOS), 3-aminopropyltrimethoxysilane, bis[3-(trimethoxysilyepropyl]-ethylenediamine, bis[3-(triethoxysilyl)propyl]-ethylenediamine, methyltrimethoxysilane (MTMS), polydimethylsilane (PDMS), propyltrimethoxysilane (PTMS), methyltriethoxysilane (MTES), ethyltriethoxysilane, dimethyldiethoxysilane, diethyldiethoxysilane, diethyldimethoxysilane, bis(3-trimethoxysilylpropyl)-N-methylamine, 3-(2-Aminoethylamino)propyltriethoxysilane, N-propyltriethoxysilane, 3-(2-Aminoethylamino)propyltrimethoxysilane, methylcyclohexyldimethoxysilane, dimethyldimethoxysilane, dicyclopentyldimethoxysilane, 3-[2(vinylbenzylamino)ethylamino]propyltrimethoxysilane, 3-aminopropyltriethoxysilane, 3-(aminopropyl)dimethylethoxysilane, 3-(aminopropyl)methyldiethoxysilane, 3-(aminopropyl)methyldimethoxysilane, 3-(aminopropyl)dimethylmethoxysilane, N-butyl-3-aminopropyltriethoxysilane, N-butyl-3-aminopropyltrimethoxysilane, N-(β-amimoethyl)-γ-amino-propyltriethoxysilane, 4-amino-butyldimethyl ethoxysilane, N-(2-Aminoethyl)-3-aminopropylmethyldimethoxysilane, N-(2-Aminoethyl)-3-aminopropylmethyldiethoxysilane, 3-aminopropylmethyldiethoxysilane, or combinations thereof. Preferably, the silane reactant includes at least one amino-containing silane reactant, more preferably bis[3-(trimethoxysilyepropyl]-ethylenediamine (enTMOS).

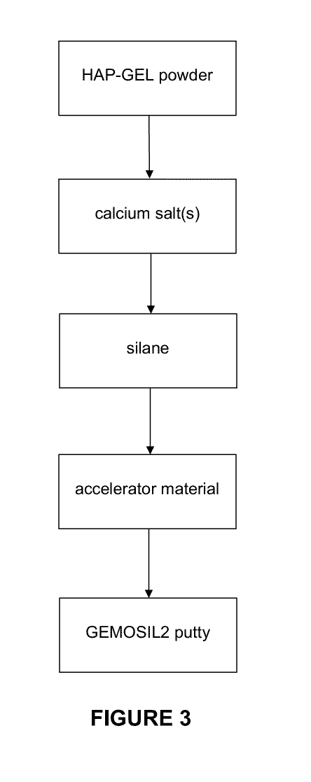

In one embodiment, at least one calcium salt, HAP-GEL powder, at least one silane, and at least one accelerator additive are combined and mixed to form the GEMOSIL2 clay, which is moldable before it hardens and is injectable to produce porous scaffolds. More specifically, Ca(OH)₂, CaO, or a mixture of Ca(OH)₂ and CaO, is combined with HAP-GEL powder to form a calcium salt(s)/HAP-GEL powder mixture. When the mixture of Ca(OH)₂ and CaO is used, the weight ratio of Ca(OH)₂/CaO of about 1:10 to about 10:1, preferably about 1:2 to about 2:1. The weight ratio of calcium salt(s)/HAP-GEL powder is about 1:10 to about 10:1, preferably about 1:2 to about 3:1. The powders are mixed to preferentially form a substantially homogeneous mixture. Thereafter, the silane, e.g., enTMOS is mixed with the calcium salt(s)/HAP-GEL powder mixture to form a second mixture. The weight/volume ratio of calcium salt/HAP-GEL powder mixture relative to silane is about 10:1 to about 0.1:1, preferably about 5:1 to about 0.5:1. Thereafter, the at least one accelerator material, preferably 1 M calcium chloride in phosphate buffer saline, is added to the second mixture to form the GEMOSIL2 putty, wherein the ratio of the second mixture/accelerator material is about 20:1 to about 1:1, preferably about 15:1 to about 10:1. The GEMOSIL2 putty will harden to form the GEMOSIL2 bioceramic. The temperature is in a range from about 15° C. to about 50° C., preferably about 20° C. to about 37° C. Before hardening, the putty can be pressed into a mold and will harden within about 5 minutes. A schematic of the method is shown in FIG. 3. It should be appreciated that the order of addition can be altered, e.g., the at least one calcium salt, the HAP-GEL powders, the at least one silane, and at least one accelerator additive can be added in any order, as readily understood by the person skilled in the art.

Although not wishing to be bound by theory, it is thought that the calcium salt, e.g., Ca(OH)₂, and the silane, e.g., enTMOS, undergo a pozzolanic reaction, similar to that that occurs in hydraulic cement (namely, pozzolan, meaning forming non-water-soluble calcium silicate hydrates (Jo B-W et al., Construction and Building Mat., 21, 1351-1355, 2006). By incorporating Ca(OH)2, it has been proven that both compressive and tensile strength of the HAP-GEL composite (HAP-GEL-CS) increased 2.1 and 4.2 times, respectively.

Advantages associated with the novel GEMOSIL2 bioceramics described herein include, but are not limited to, compatibility with carbon-based lifeforms, excellent mechanical strength, better elasticity than conventional bioglass, excellent compressive strength, superb formability for scaffolding, a moldable composite putty which is fast setting, a water resistant material, and upregulated cell differentiation.

Optionally, other components or additives may be added to the formable bioceramic. These additives may be added for various reasons. For example, additives may be added to increase biocompatibility, to decrease the possibility of rejection, to decrease the risk of infection, to increase the rate of natural bone growth in the bioceramic, increase tensile strength to achieve the mechanical quality index of natural bone, or to increase the rate of natural cell growth near the implant. Additives may also be added to change or enhance some of the properties of the bioceramic. For example, the bioceramic may include long chain polymers, growth factors, cells, other materials and elements, curing or hardening components, and other possible additives.

The GEMOSIL2 bioceramic may be used for a wide range of alloplastic uses, for a variety of purposes, and in a variety of applications. Alloplastic refers to synthetic biomaterials, in contrast to natural biomaterials which may be from the same individual (autogenic), from the same species (allogenic), or from a different species (xenogenic). The properties of the GEMOSIL2 bioceramic may be modified to better meet the requirements of the use, purpose, or application for which it is intended. The properties depend in part on the gelatin used, the stoichiometry of the HAP-GEL, the amount and type of silane reactant(s) used, the calcium salts used, the accelerator materials, and the stoichiometry of the components of the GEMOSIL2 bioceramic. Thus, the resulting bioceramic may have a wide range of mechanical properties.

These various properties lead to the ability of the GEMOSIL2 bioceramic to be used in a wide range of tissue engineering applications. For example, the GEMOSIL2 bioceramic can be made in scaffolds, which can deliver cells, growth factors, and other additives to a healing site. This can be used to regenerate bone, cartilage, and other tissues. Nano-scaled microstructures can be used to promote cell attachment, growth, and differentiation. Alternatively, the GEMOSIL2 bioceramic may be used to engineer alloplastic grafts. Thus, tissue engineering may be used to replace or augment many natural body tissues. Tissues may be regenerated using these types of structures, and additives may be used to compensate for deficiencies in the patient. Other structures that promote the rapid integration of the GEMOSIL2 bioceramic with the natural tissues may also be used effectively. For example, a structure of the GEMOSIL2 bioceramic may be implanted into a bone, which then acts to stimulate bone regeneration, especially in critical size defects in craniofacial and other skeletal areas. As another example, the GEMOSIL2 bioceramic may be implanted for cartilage replacement, which may stimulate cartilage regeneration.

The GEMOSIL2 bioceramic may be produced in different forms, depending upon the intended use and purpose. Suitable forms include solid, putty, paste, and liquid. If the GEMOSIL2 bioceramic is in solid form, it may be, for example, a shaped or unshaped solid, it may be a pre-formed solid, it may be a frame or a lattice, or another solid form. The GEMOSIL2 bioceramic may be formed into a porous scaffold. The solid form may be very stiff, stiff, slightly flexible, soft, rubbery, or other. The GEMOSIL2 bioceramic may be a putty. If in putty form, it may be anywhere from a dense or thin putty. The GEMOSIL2 bioceramic may be a paste. If a paste, it may be anywhere from a thick to a thin paste. If a liquid, it may be from very viscous to very thin.

Uses of the GEMOSIL2 bioceramic include, but are not limited to: for bones, such as for bone graft material, bone cement, or bone replacement; for dental procedures, such as for dental implants, fillings, jaw strengthening or tooth replacement; for joint replacement; for cartilage replacement or reinforcement; for tendon or ligament replacement or repair; and a wide range of tissue engineering applications, including assisting in regenerating bodily tissues.

Additionally, the compressive strength of the GEMOSIL2 bioceramic and various natural bones may be tested and compared. A GEMOSIL2 bioceramic may have compressive strength comparable to that of natural bone.

In a fourth aspect, a GEMOSIL2 bioceramic is disclosed, said bioceramic being produced using the method of the third aspect. The GEMOSIL2 bioceramic comprises silane and HAP-GEL nanocrystals and can harden in water. The compressive strength of the GEMOSIL2 bioceramic is preferably in a range from about 80 MPa to about 170 MPa.

### EXAMPLE 1

A 100 mg sample of HAP-GEL powder was transferred into a mortar and grinded into fine powder. An amount of calcium hydroxide/calcium oxide powder as shown in Table 1 was added into the mortar and mixed with the HAP-GEL powder for 2 minutes. Then, the amount of enTMOS as shown in Table 1 was added and the mixture was continuously blended for 30 seconds. This mixture appeared uniformly yellow in color. To convert the mixture into putty, calcium chloride solution (1M in phosphate buffer saline, PBS 1×) was added to the mixture and mixed until the sample showed plasticity. The sample appeared as putty and was pressed with a mold to create round shape disc samples and cylindrical shape samples. All samples solidified within 5 minutes.

TABLE 1
Amounts of components used to make GEMOSIL2 bioceramic.

| | HAP-GEL | enTMOS | Ca(OH)₂ | CaO | 1M CaCl₂ |
|---|---|---|---|---|---|
| Samples | (mg) | (μL) | (mg) | (mg) | (μL) |
| 1 | 100 | 300 | 0 | 0 | 48 |
| 2 | 100 | 300 | 100 | 0 | 48 |
| 3 | 100 | 300 | 200 | 0 | 48 |
| 4 | 100 | 400 | 0 | 100 | 64 |
| 5 | 100 | 400 | 0 | 200 | 64 |

The GEMOSIL2 bioceramic has higher strength both during hardening (2 hours setting in water, 42 MPa) and fully dry (100.1 Ma) than that of GEMOSIL (e.g., made according to U.S. patent application Ser. No. 12/685,743). Moreover, the GEMOSIL2 bioceramic contains Ca(OH)₂, which is known to encourage bone growth. In addition, the GEMOSIL2 bioceramic also showed 100 times greater three-point bending strength than GEMOSIL.

### EXAMPLE 2

A 100 mg sample of HAP-GEL powder was transferred into a mortar and ground into fine powder. The predetermined amount of calcium hydroxide/calcium oxide powder (in a 1:1 ratio) as shown in Table 2 was added into the mortar and mixed with HAP-GEL powder for 2 minutes. Then, the predetermined amount of dopamine in 100-400 μL HCl solution(25% 1N in methanol) was added and the mixture was continuously blended for 60-120 seconds. This mixture turned brown in color. To convert the mixture into putty, ammonia persulfate (25%-60% in DI water) was added to the mixture and mixed until the sample showed plasticity within 5-10 minutes. The sample appeared as putty and was pressed with a mold to create custom shaped samples. All samples were dehydrated in air at room temperature.

TABLE 2
Amounts of components used to make PDHG
nanocomposite (GEMUSSEL).

| | HAP-GEL | Ca(OH₂/ | Dopamine in | ammonium |
|---|---|---|---|---|
| Samples | (mg) | CaO (mg) | HCl (mg) | persulfate (μL) |
| 1 | 100 | 50 | 50 | 24 |
| 2 | 100 | 100 | 100 | 54 |
| 3 | 100 | 200 | 200 | 108 |
| 4 | 100 | 300 | 300 | 144 |

TABLE 3
Amounts of components used to make PDHG
nanocomposite (GEMUSSEL).

| | | | | | Ammonium |
|---|---|---|---|---|---|
| | HAP-GEL | Ca(OH)2 | Dopamine | enTMOS | persulfate |
| Samples | (mg) | (mg) | (mg) | (μL) | (μL) |
| 1 | 100 | 100 | 50 | 250 | 40 |
| 2 | 150 | 100 | 50 | 250 | 40 |
| 3 | 100 | 100 | 10 | 250 | 40 |
| 4 | 150 | 100 | 10 | 250 | 40 |
| 5 | 50 | 100 | 50 | 250 | 40 |
| 6 | 50 | 100 | 50 | 250 | 60 |

The viscosity of the PDHG paste was found to be controlled by water present in the components of the mixtures. By controlling the amounts of total water, a viscous paste was successfully formulated that would gradually thicken (5-10 minutes) and reach a consistency that was injectable. In a pilot test, the 1 cc syringe with PDHG was hand-pressed to fabricate a porous scaffold plate for rat calvarium bone replacement. As shown in FIG. 3B, the material became thixotropic and injectable by loading into a 1 cc syringe. Upon drying, the nanocomposite has a two-layer scaffold (see FIG. 3C) that maintains its structure and integrity even after being immersed in water for 1 hour (see, FIG. 3D).

### EXAMPLE 3

Three groups of 35 mm dishes containing GEMOSIL2 with and without dopamine and no coating were tested for preosteoblast (MC3T3-E1) cell culture. Cytoskeleton and adhesive focal spots were assessed by phalloidin staining and vinculin immunofluorescent staining at day 3. For proliferation assay after 1, 3, 5, 7, 14, and 21 days in culture, 3-(4,5-dimethylthiazol-2-yl)-5-(3-carboxymethoxy-phenyl)-2-(4-sulfophenyl)-2H-tetrazolium salt (MTS) reagent (Promega, Madison, Wis., USA) was used to measure relative cell numbers based on the concentration of the formazan production. Coated dishes without cells were used as a blank (negative control). Collagen formation and mineralization were confirmed by Alizarin red and picrosirious stains, respectively. It was concluded that the incorporation of polydopamine in the substrate increased initial cellular adhesion and spreading, proliferation, and differentiation.

### EXAMPLE 4

MC3T3-E1 cells were cultured on PDHG coated and control (no-coating) 35 mm dishes using the osteogenic medium. At 4 and 7 days, the mRNA and protein expression for dopamine receptors were harvested for qRT-PCR and western blot analysis, respectively. Undifferentiated human mesenchymal stem cells (hMSC) were also analyzed by western blot. Our data showed that both DrD1 and DrD3 receptors were abundantly expressed in differentiated MC3T3-E1 cells, but not in undifferentiated hMSC cells. This is thought to be the first finding that osteoblasts also have receptors for neurotransmitter dopamine. Our data also showed there was no tyrosine-hydroxylase expression, indicating that there is no endogenous dopamine production. It is surprising that dopamine treatment induced a decrease in OPTN expression. OPTN is known to be an apoptosis regulator upstream of NF-kB pathway in neuron cells, and recently found to related with Paget's' disease. This suggests that exogenous dopamine may directly promote proliferation and/or inhibiting apoptosis by inhibiting OPTN expression.

Accordingly, while the invention has been described herein in reference to specific aspects, features and illustrative embodiments of the invention, it will be appreciated that the utility of the invention is not thus limited, but rather extends to and encompasses numerous other aspects, features and embodiments that result from the adsorption-induced tension in molecular (chemical and physical) bonds of adsorbed macromolecules and macromolecular assemblies. Accordingly, the claims hereafter set forth are intended to be correspondingly broadly construed, as including all such aspects, features and embodiments, within their spirit and scope.

1. A method of making a GEMUSSEL nanocomposite comprising combining hydroxyapatite-gelatin (HAP-GEL) bioceramic, at least one dopamine species, at least one oxidizing agent, and at least one calcium salt to form a GEMUSSEL material, wherein the GEMUSSEL material hardens to form the GEMUSSEL nanocomposite.

2. The method of claim 1, wherein water is combined with the HAP-GEL bioceramic, at least one dopamine species, at least one oxidizing agent, and at least one calcium salt to form the GEMUSSEL material.

3. The method of claim 1, wherein at least one silane reactant is added with the HAP-GEL bioceramic, at least one dopamine species, at least one oxidizing agent, and at least one calcium salt to form the GEMUSSEL material.

4. The method of claim 3, wherein the HAP-GEL bioceramic, the at least one dopamine species, and the at least one calcium salt are mixed to form mixture 1;
the at least one silane reactant is combined with mixture 1 to form mixture 2; and
the at least one oxidizing agent is combined with mixture 2 to form the GEMUSSEL material.

5. (canceled)

6. The method of claim 1, wherein the calcium salts comprise a species selected from the group consisting of calcium oxide, calcium hydroxide, calcium carbonate, calcium nitrate, calcium phosphate, calcium fluoride, calcium chloride, calcium iodide, calcium oxalate, calcium citrate, calcium pyrophosphate, and combination thereof.

7. (canceled)

8. The method of claim 1, wherein the oxidizing agent comprises a species selected from the group consisting of hydrogen peroxide (H₂O₂), ferric nitrate (Fe(NO₃)₃), potassium iodate (KIO₃), nitric acid (HNO₃), ammonium chlorite (NH₄ClO₂), ammonium chlorate (NH₄ClO₃), ammonium iodate (NH₄IO₃), ammonium perborate (NH₄BO₃), ammonium perchlorate (NH₄ClO₄), ammonium periodate (NH₄IO₃), ammonium persulfate ((NH₄)₂S₂O₈), tetramethylammonium chlorite ((N(CH₃)₄)ClO₂), tetramethylammonium chlorate ((N(CH₃)₄)ClO₃), tetramethylammonium iodate ((N(CH₃)₄)IO₃), tetramethylammonium perborate ((N(CH₃)₄)BO₃), tetramethylammonium perchlorate ((N(CH₃)₄)ClO₄), tetramethylammonium periodate ((N(CH₃)₄)IO₄), tetramethylammonium persulfate ((N(CH₃)₄)S₂O₈), urea hydrogen peroxide ((CO(NH₂)₂)H₂O₂), and combinations thereof.

9. (canceled)

10. The method of claim 1, wherein the at least one dopamine species is 4-(2-aminoethyl)benzene-1,2-diol.

11. (canceled)

12. The method of claim 1, wherein the dopamine is dissolved in an acid/alcohol solvent.

13. The method of claim 3, wherein the at least one silane reactant comprises a species selected from the group consisting of tetramethylorthosilicate (TMOS), tetraethylorthosilicate (TEOS), 3-aminopropyltrimethoxysilane, bis[3-(trimethoxysilyl)propyl]-ethylenediamine, bis[3-(triethoxysilyl)propyl]-ethylenediamine, methyltrimethoxysilane (MTMS), polydimethylsilane (PDMS), propyltrimethoxysilane (PTMS), methyltriethoxysilane (MTES), ethyltriethoxysilane, dimethyldiethoxysilane, diethyldiethoxysilane, diethyldimethoxysilane, bis(3-trimethoxysilylpropyl)-N-methylamine, 3-(2-Aminoethylamino)propyltriethoxysilane, N-propyltriethoxysilane, 3-(2-Aminoethylamino)propyltrimethoxysilane, methylcyclohexyldimethoxysilane, dimethyldimethoxysilane, dicyclopentyldimethoxysilane, 3-[2(vinylbenzylamino)ethylamino]propyltrimethoxysilane, 3-aminopropyltriethoxysilane, 3-(aminopropyl)dimethylethoxysilane, 3-(aminopropyl)methyldiethoxysilane, 3-(aminopropyl)methyldimethoxysilane, 3-(aminopropyl)dimethylmethoxysilane, N-butyl-3-aminopropyltriethoxysilane, N-butyl-3-aminopropyltrimethoxysilane, N-(β-amimoethyl)-γ-amino-propyltriethoxysilane, 4-amino-butyldimethyl ethoxysilane, N-(2-Aminoethyl)-3-aminopropylmethyldimethoxysilane, N-(2-Aminoethyl)-3-aminopropylmethyldiethoxysilane, 3-aminopropylmethyldiethoxysilane, and combinations thereof.

14. The method of claim 3, wherein the at least one silane reactant comprises bis[3-(trimethoxysilyl)propyl]-ethylenediamine (enTMOS).

15. The method of claim 1, wherein the temperature is about 15° C. to about 50° C.

16. The method of claim 1, wherein the GEMUSSEL material is a paste or a putty or a solid or is injectable.

17.-20. (canceled)

21. A polydopamine bio-inspired hydroxyapatite-gelatin nanocomposites (PDHG) nanocomposite comprising dopamine and hydroxyapatite-gelatin nanocrystals.

22. A method of making an aminosilica-based hydroxyapatite-gelatin bioceramic, said method comprising combining hydroxyapatite-gelatin (HAP-GEL) bioceramic, at least one silane reactant, at least one calcium salt, and at least one accelerator material to form the GEMOSIL2 material, wherein the GEMOSIL2 material hardens to form the GEMOSIL2 bioceramic.

23. The method of claim 22, wherein the HAP-GEL comprises bis[3-(trimethoxysilyl)propyl]-ethylenediamine (enTMOS).

24. The method of claim 22, wherein the accelerator materials comprise a species selected from the group consisting of poly(lactic-co-glycolic acid), poly(lactic acid), poly(glycolic acid), polyacrylic acid, poly(ethylene oxide), calcium phosphate, potassium chloride, calcium carbide, calcium chloride, sodium chloride, polystyrene, and combinations thereof.

25. (canceled)

26. The method of claim 22, wherein the at least one calcium salt comprises a species selected from the group consisting of calcium oxide, calcium hydroxide, calcium carbonate, calcium nitrate, calcium phosphate, calcium fluoride, calcium chloride, calcium iodide, calcium oxalate, calcium citrate, calcium pyrophosphate, and combinations thereof.

27. The method of claim 22, wherein the at least one silane reactant comprises a species selected from the group consisting of tetramethylorthosilicate (TMOS), tetraethylorthosilicate (TEOS), 3-aminopropyltrimethoxysilane, bis[3-(trimethoxysilyl)propyl]-ethylenediamine, bis[3-(triethoxysilyl)propyl]-ethylenediamine, methyltrimethoxysilane (MTMS), polydimethylsilane (PDMS), propyltrimethoxysilane (PTMS), methyltriethoxysilane (MTES), ethyltriethoxysilane, dimethyldiethoxysilane, diethyldiethoxysilane, diethyldimethoxysilane, bis(3-trimethoxysilylpropyl)-N-methylamine, 3-(2-Aminoethylamino)propyltriethoxysilane, N-propyltriethoxysilane, 3-(2-Aminoethylamino)propyltrimethoxysilane, methylcyclohexyldimethoxysilane, dimethyldimethoxysilane, dicyclopentyldimethoxysilane, 3-[2(vinylbenzylamino)ethylamino]propyltrimethoxysilane, 3-aminopropyltriethoxysilane, 3-(aminopropyl)dimethylethoxysilane, 3-(aminopropyl)methyldiethoxysilane, 3-(aminopropyl)methyldimethoxysilane, 3-(aminopropyl)dimethylmethoxysilane, N-butyl-3-aminopropyltriethoxysilane, N-butyl-3-aminopropyltrimethoxysilane, N-(β-amimoethyl)-γ-amino-propyltriethoxysilane, 4-amino-butyldimethyl ethoxysilane, N-(2-Aminoethyl)-3-aminopropylmethyldimethoxysilane, N-(2-Aminoethyl)-3-aminopropylmethyldiethoxysilane, 3-aminopropylmethyldiethoxysilane, and combinations thereof.

28. (canceled)

29. The method of claim 22, wherein the temperature is in a range from about 15° C. to about 50° C.

30. The method of claim 22, wherein the GEMOSIL2 material is a paste or a putty or a solid or is injectable.

31.-33. (canceled)
